(19) United States
(12) Patent Application Publication
(10) Pub. No.: US 2026/0034167 A1
(43) Pub. Date: Feb. 5, 2026

(54) HYALURONIC ACID-BASED HYDROGELS FOR JOINT THERAPY

(71) Applicants: The United States Government as represented by the Department of Veterans Affairs, Washington, DC (US); The Trustees of the University of Pennsylvania, Philadelphia, PA (US); Emory University, Atlanta, GA (US)

(72) Inventors: Jay Milan Patel, Atlanta, GA (US); Robert L Mauck, Philadelphia, PA (US); Tristan Pepper, Atlanta, GA (US); Hanna Solomon, Atlanta, GA (US)

(21) Appl. No.: 19/288,964

(22) Filed: Aug. 1, 2025

Related U.S. Application Data
(60) Provisional application No. 63/678,749, filed on Aug. 2, 2024.

(51) Int. Cl. A61K 31/728; A61K 9/00; A61K 9/08; A61K 41/00; A61K 47/54; A61K 47/64; A61P 19/02

(52) U.S. Cl. CPC A61K 31/728; A61K 9/0019; A61K 9/08; A61K 41/00; A61K 47/545; A61K 47/64; A61P 19/02

(57) ABSTRACT

Hyaluronic acid-based polymers which can crosslink within joint tissue either by photo-crosslinking or chemical crosslinking to provide a hydrogel within the joint tissue to aid with joint tissue protection, regeneration, and repair.

### CROSS-REFERENCE TO RELATED APPLICATIONS

This application claims priority to U.S. Provisional Application No. 63/678,749, filed Aug. 2, 2024, the entirety of which is incorporated into this application by reference.

### REFERENCE TO SEQUENCE LISTING

The Sequence Listing submitted Aug. 1, 2025 as a text file named “37759.0727U1.xml,” created on Aug. 1, 2025, and having a size of 17,453 bytes is hereby incorporated by reference pursuant to 37 C.F.R. § 1.52 (c) (5).

### BACKGROUND

Joint tissue is prone to degeneration over time and with age. In some cases, osteoarthritis can form, which is a debilitating condition that causes pain, discomfort, and disability. The disease often originates with a traumatic injury to the joint, such as a ligament or meniscus tear, ankle fracture, or cartilage injury. The most common treatments for early arthritis are non-steroidal anti-inflammatoires, corticosteroids, and hyaluronic acid injections. All these treatments aim to reduce inflammation in the joint, yet they have a short residence time, often necessitating frequent injections while leaving symptoms unresolved. Furthermore, these treatments do not impact tissue changes that have occurred early in the arthritic process.

Similarly, cartilage repair strategies offer short-term relief but leave the repair tissue and surrounding cartilage susceptible to wear, leading to arthritic progression. Two of the significant issues with current approaches are inadequate defect fill due to early contraction and the propensity for repairs to undergo fibrosis, resulting in mechanically inferior tissue. The most common repair technique is microfracture (MFx), which involves puncturing the subchondral bone to recruit regenerative marrow elements. Unfortunately, the marrow clot experiences both rapid contraction and fibrosis, inhibiting volumetric and functional cartilage regeneration. Current clinical strategies to augment the MFx environment involve implantation of a scaffold or sponge. Examples include Chondrogide (collagen), BST-CarGel (chitosan), BioCartilage (micronized cartilage), GelrinC (PEGDA), and Hyalofast (benzyl ester of hyaluronic acid). However, all these scaffolds require fixation and do not allow the clot to form. Thus, treatments and prophylactic measures that effectively promote volumetric and functional cartilage protection and repair would be groundbreaking in cartilage injury management.

### SUMMARY

The disclosed methods and compositions utilize a joint tissue-penetrating system that infiltrates into multiple joint tissues, providing a protective effect and restoring the biology and immediate microenvironment of the cells. Results indicate that the described hyaluronic acid polymers, once formed into hydrogels, can restore the biology of joint tissue cells and the joint environment, and can expand the protective effect from cartilage and other joint tissues.

The method of protecting or improving the quality of joint tissue in a subject comprises a) applying an aqueous solution of a methacrylated hyaluronic acid polymer to the joint tissue of the subject, wherein the methacrylated hyaluronic acid polymer has the structure:

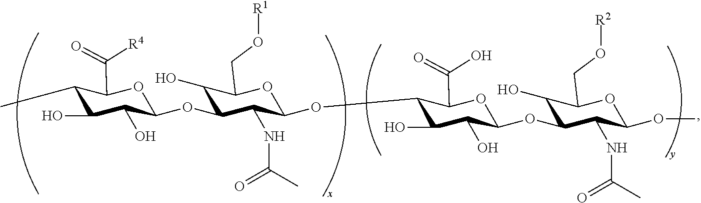

wherein x and y are independently integers ranging from 5 to 50; each R¹ substituent is hydrogen or a methacrylate group having the structure:

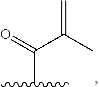 [SMILES: *C(=O)C(=C)C]

wherein at least some of the R¹ substituents are the methacrylate group; and each R² substituent is hydrogen or a ligand comprising a peptide; b) allowing the aqueous solution to diffuse into the joint tissue; and c) photo-crosslinking the methacrylated hyaluronic acid polymer to form a hydrogel within the joint tissue.

The method of repairing damaged articular cartilage in joint tissue of a subject comprises a) microfracturing a portion of the subchondral bone plate beneath a defect area of the damaged articular cartilage and allowing a fibrin clot to form in the defect area; b) applying an aqueous solution of an azide-modified hyaluronic acid polymer to the joint tissue of the subject, wherein the azide-modified hyaluronic acid polymer has the structure:

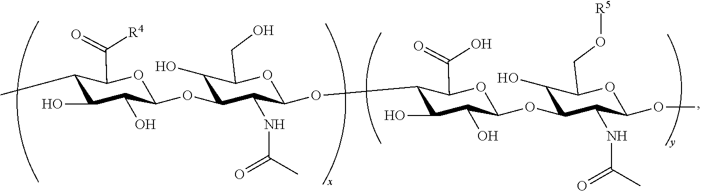

wherein x and y are independently integers ranging from 5 to 50 (representing repreating units);
each R⁴ substituent is-OH or an azide group having the structure:

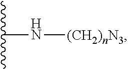

wherein n is an integer ranging from 1-10, wherein at least some of the R⁴ substituents are the azide group, and each R⁵ substituent is hydrogen or a ligand comprising a peptide; c) allowing the aqueous solution to diffuse into the defect area of the damaged articular cartilage; d) applying a solution of a crosslinker to the joint tissue of the subject, wherein the crosslinker has at least two alkyne functional groups; and e) allowing the azide-modified hyaluronic acid polymer to react with the alkyne functional groups of the crosslinker via cycloaddition within the joint tissue to form a hydrogel.

Also described are compositions, systems, and kits that include the hyaluronic acid polymers and, in some examples, a crosslinking tool such as a light source or a chemical crosslinker.

### DETAILED DESCRIPTION

The tissue-penetrating approach described below allows for in-tissue delivery of both hyaluronic acid polymers and in some examples, cell-instructive and pro-regenerative peptides which can be conjugated to the polymers. The polymers can be delivered to joint tissue and crosslinked within the tissue, for example by photocrosslinking or chemical crosslinking. Such a system provides many advantages over current joint health therapies. First, the hydrogels can reinforce the joint tissue mechanically to provide additional support to damaged cartilage (or prevent degeneration of healthy cartilage). Second, the hydrogels can provide delivery of and promote additional synthesis of hyaluronic acid and anti-inflammatory or pro-healing peptides within the tissue, which offers advantages over traditional injections that localize within the joint fluid and are typically cleared within a day. Third, the hydrogel systems localize to the immediate cellular environment within the joint, providing direct cues to cartilage cells to produce additional hyaluronic acid and restore cellular environment. Finally, the hydrogel systems can infiltrate within the synovium tissues of the joint and tissues surrounding the joint, reducing fibrosis and joint-degrading processes. Thus, the hyaluronic acid-based gel systems can serve as an all-in-one joint treatment for preservation, healing, and repair of joint tissue, including during early phases of arthritis and following traumatic injuries.

The disclosed methods are useful for protecting or improving the quality of joint tissue, including protecting against degeneration, as well as repairing damaged articular cartilage in joint tissue. The methods can be carried out on any living subject with jointed limbs, including humans and animals, such as veterinary animals (e.g., dogs, cats, and horses).

The prophylactic and joint repair methods involve applying a hyaluronic acid polymer to the joint tissue of the subject and crosslinking the polymer or allowing the polymer to crosslink to form a hydrogel. The term “joint tissue” refers to any tissue in any type of joint, including synovial joints, which are movable joints characterized by a joint cavity filled with synovial fluid. Synovial joints include ball-and-socket joints such as the shoulder and hip joint, hinge joints such as the elbow joint, knee joint, joints of the fingers and toes, pivot joints such as the atlantoaxial joint between the first and second cervical vertebrae and proximal radioulnar joint of the forearm, condyloid (ellipsoid) joints such as the wrist joint and metacarpophalangeal joints, saddle joints such as the carpometacarpal joint of the thumb, and gliding joints such as the intercarpal joints in the wrist. The term “joint tissue” also refers to cartilaginous joints, including synchondroses, such as epiphyseal plates, and symphyses, including fibrocartilage such as pubic symphysis and intervertebral discs. The methods may not be useful for subjects with end-stage osteoarthritis where the joint no longer includes any cartilage.

The methods are particularly useful for protecting, improving the quality of, or repairing synovial joints such as the knee. The knee, for example, includes articular cartilage, which is composed of smooth tissue covering the ends of the femur, tibia, and back of the patella. Articular cartilage of the knee is prone to degeneration and is often injured. The crosslinked hydrogel can reinforce damaged (or healthy) articular cartilage and prevent inflammation-induced degeneration. The hydrogels can also integrate with other tissues in the joints and provided them with a biologically protective effect, including inducing the production of more hyaluronic acid and restoring or protecting the microenvironment around cells in articular cartilage. The hydrogels can diffuse into the surrounding synovium and prevent inflammatory activity, fibrosis, and restore natural hyaluronic acid production. In addition, some disclosed methods involve the repair of damaged joint tissue and include a microfracturing step. The disclosed hydrogels may infiltrate the fibrin-rich clot that forms after microfracturing to allow for more efficient healing.

The hydrogels can have positive effects on the microcellular environment within the joint. For instance, the hydrogels can have effects on chondrocytes, which are cells found in cartilage tissue that are responsible for maintaining and synthesizing the extracellular matrix. Chondrocytes are crucial for the growth and repair of cartilage. Unlike most other tissues, cartilage is avascular, which means chondrocytes rely on diffusion to receive nutrients and remove waste products, making cartilage repair a slow process. Chondrocytes undergo changes as cartilage ages or becomes damaged, which can lead to conditions like osteoarthritis.

The methods can protect or improve the quality of healthy joint tissue in the subject. Quality of joint tissue can be assessed using various clinical evaluations, imaging, and laboratory analysis. Common methods include examination by a healthcare profession to assess joint function, range of motion, swelling, and pain, including palpitation and specific movement tests. Imaging techniques include x-ray, magnetic resonance imaging (MRI), and ultrasound. Laboratory tests include determining the presence of biochemical markers such as oligomeric matrix protein (COMP) and cytokines, which can indicate inflammation or cartilage degradation, and histological analysis obtained from biopsy. Various functional tests are also useful for determining the quality of joint tissue, including gait analysis and minimally invasive procedures such as arthroscopy.

### Methods of Protecting, Improving, and Repairing Joint Tissue

### Methods Using Photo-Crosslinking

One example of the method involves protecting or improving the quality of joint tissue in a subject by cross-linking a hydrogel precursor within the joint tissue using photo-crosslinking. The method comprises applying an aqueous solution of a methacrylated hyaluronic acid polymer to the joint tissue of the subject. Because photo-crosslinking is used in this method, the aqueous solution of the methacrylated hyaluronic acid polymer can be applied to the joint tissue during an open joint surgery, where an incision is made on the joint and interior joint tissues are exposed. The method can also be used with minimally invasive procedures such as arthroscopy, which can involve the use of an arthroscopic light probe for photo-crosslinking.

The methacrylated hyaluronic acid polymer has the structure:

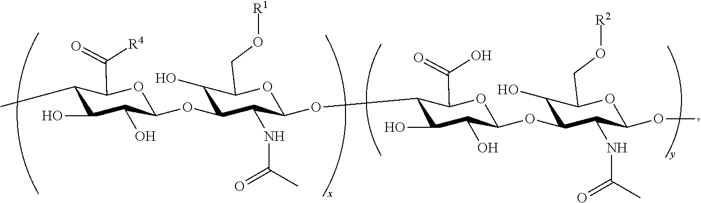

where x and y are independently integers ranging from 5 to 50. The units x and y correspond to molecular weights of the methacrylated hyaluronic acid polymer of 3,000 kDa to 50,000 kDa, e.g., 10,000 kDa to 40,000 kDa, or about 20,000 kDa (where the term “about” indicates the stated value plus and minus 20% of the stated value). The term “molecular weight” refers to weight-average molecular weight (Mw), determined by size exclusion chromatography (SEC).

Each R¹ substituent is hydrogen or a methacrylate group having the structure:

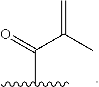 [SMILES: *C(=O)C(=C)C]

At least some of the R¹ substituents are the methacrylate group, which allows for the hyaluronic acid polymer to be photo-crosslinked once applied to the joint tissue. The methacrylated hyaluronic acid polymer can have a 20% to 100% degree of methacrylation. The “degree of methacrylation” means the percentage of hyaluronic acid monomer residues in the polymer that include methacrylated groups.

Each R² substituent is hydrogen or a ligand comprising a peptide. In some examples, each R² is hydrogen. In other examples, at least some of the R² substituents are the ligand comprising the peptide. The peptide can be a cell-instructive peptide or a pro-regenerative peptide having from 3-100 (e.g., 3-80, 3-50, 30-20, 10-20, 10-12) amino acids, as discussed in more detail below. The ligand comprising the peptide can have the structure:

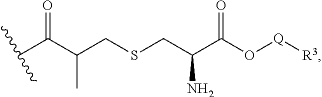

where Q is an optional spacer, and R³ is the peptide. Q in some instances is present in the ligand and has the structure:

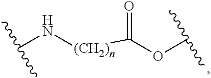

where n is an integer ranging from 1 to 10, e.g., 2-8, 3-6, or 5. The methacrylated hyaluronic acid polymer can be covalently attached to the peptide by the addition of a terminal cysteine residue on the ligand (optionally separated from the peptide by a spacer, Q), which can react with methacrylate groups on the polymer through thiol-Michael Addition.

The methacrylated hyaluronic acid polymer can be present in the aqueous solution at a concentration of 1-10% weight by volume. For instance, methacrylated hyaluronic acid polymer can be present in the aqueous solution at a concentration of 1-10%, 1-8%, 1-5%, or about 4% (where “about” means plus and minus 10% of the stated value) weight by volume.

Once applied to the joint tissue, the aqueous solution can be allowed to diffuse into the joint tissue, in some cases throughout the joint tissue, prior to photo-crosslinking the polymer. Diffusion of the polymer into and throughout the joint tissue can take up to 20 minutes, e.g., 5-10 minutes. After this time, the methacrylated hyaluronic acid polymer is photo-crosslinked at the methacrylate groups to form a hydrogel within the joint tissue.

Photocrosslinking the methacrylated hyaluronic acid polymer can be accomplished within the joint tissue by exposing the tissue to various wavelengths of light, including UV light (e.g., UVA-315-400 nm, UVB-280-315 nm, UVC-100-280 nm), visible light, blue light (e.g., 400-500 nm, or about 405 nm) and infrared or near-infrared light. In the case of visible light and infrared or near-infrared light, or if desired with UV crosslinking, a photoinitiator can be used. Examples include Irgacure 2959 (2-Hydroxy-4′-(2-hydroxyethoxy)-2-methylpropiophenone), which is effective in the UV range typically around 295 nm, Lithium Phenyl (2,4,6-Trimethylbenzoyl) Phosphinate (LAP), which can be activated by visible light around 365-405 nm, VA-086 (2,2′-Azobis [2-methyl-N-(2-hydroxyethyl) propionamide]), which is active in the UV range, and Camphorquinone, which is active in the visible range around 450 nm. Photo-crosslinking can be accomplished over a suitable amount of time (e.g., 1-10 minutes).

The photo-crosslinking methods using the methacrylated hyaluronic acid polymer can also be used for repairing damaged articular cartilage in joint tissue of a subject. In one example, these methods involve a microfracture (MFx) technique prior to applying the solution of the methacrylated hyaluronic acid polymer to the joint tissue. The microfracture technique is a minimally invasive surgical procedure used to repair damaged articular cartilage in joints, most commonly the knee, but also the ankle, hip, and shoulder. It is a marrow-stimulation technique designed to promote the growth of new cartilage-like tissue to fill in areas of cartilage defect or loss. The methacrylated hyaluronic acid polymer can also be applied to articular cartilage, joint synovium, the meniscus, and any other joint tissue for repairing, preserving, or protecting joint tissue.

Prior to applying the methacrylated hyaluronic acid polymer to the joint tissue, loose or damaged cartilage within the joint can be removed, and edges of the defect can be cleaned to create a suitable environment for healing. Multiple small holes or microfractures can then be made in the subchondral bone plate using an awl or pick for example. These microfractures allow bone marrow elements (including mesenchymal stem cells, growth factors, and blood) to flow into the defect. Thereafter, a fibrin clot forms in the defect area, acting as a scaffold for fibrocartilage growth and healing through the formation of fibrocartilaginous tissue. The disclosed hydrogels can improve this healing process, for example, by infiltrating the cracks of the microfractures. The methacrylated hyaluronic acid polymer can also be applied on top of a formed fibrin clot or can be mixed within the clot and subsequently photo-crosslinked.

### Methods Using Chemical Crosslinking

Methods using chemical crosslinking can be used in the same manner as described above. These methods can involve protecting or improving the quality of joint tissue in a subject by applying an aqueous solution of an azide-modified hyaluronic acid polymer to the joint tissue, allowing the azide-modified hyaluronic acid polymer to diffuse into and throughout the joint tissue, followed by applying a crosslinker having at least two alkyne functional groups to the joint tissue, and allowing the azide-modified hyaluronic acid polymer and the crosslinker to react via cycloaddition (Click chemistry) within the joint tissue to form the hydrogel. One advantage of the chemical crosslinking methods is that they may be performed with minimally invasive surgical procedures such as arthroscopic surgery or simply by intra-articular injection of the azide-modified hyaluronic acid polymer and crosslinker. Photo-crosslinking is not required and thus opening the joint or accessing the joint arthroscopically is not necessary when using the chemical crosslinking systems.

Chemical crosslinking can also be used as part of methods of repairing damaged articular cartilage in joint tissue of a subject. As described above, before applying the azide-modified hyaluronic acid polymer and crosslinker to the joint tissue, a portion of the subchondral bone plate beneath a defect area of damaged articular cartilage can be microfractured, and a fibrin clot allowed to form in the defect area. The azide-modified hyaluronic acid polymer can then be applied to the joint tissue and allowed to diffuse into and throughout the tissue including into the defect area of the cartilage where the fibrin clot has formed. After diffusion of the azide-modified hyaluronic acid polymer, the crosslinker can be applied to the joint tissue and similarly allowed to diffuse and react with the azide groups of the polymer. This forms a crosslinked hyaluronic acid polymer within the joint tissue including within the fibrin clot itself, which improves the healing process as already explained. The azide-modified hyaluronic acid polymer can also be applied on top of a formed fibrin clot or can be mixed within the clot and subsequently crosslinked. The azide-modified hyaluronic acid polymer can also be applied to articular cartilage, joint synovium, the meniscus, and any other joint tissue for repairing, preserving, or protecting joint tissue.

The azide-modified hyaluronic acid polymer has the structure:

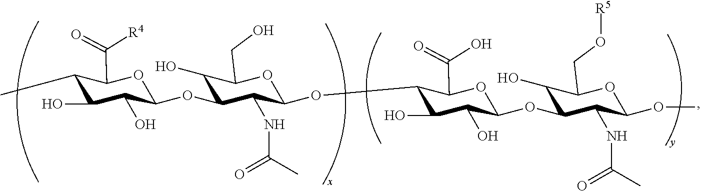

where x and y are independently integers ranging from 5 to 50, generally corresponding to a weight-average molecular weight (Mw) as determined by SEC of 3,000 kDa to 50,000 kDa, e.g., 10,000 kDa to 40,000 kDa, or about 20,000 kDa (where the term “about” indicates the stated value plus and minus 20% of the stated value).

Each R⁴ substituent is-OH or an azide group having the structure:

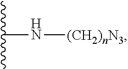

where n is an integer ranging from 1-10, e.g., 2-8, 3-6, or 4. At least some of the R⁴ substituents are the azide group, which undergoes chemical crosslinking with the alkyne functionalized crosslinker through Click chemistry. The azide-modified hyaluronic acid polymer can have a 20% to 100% degree of azide modification, defined as the percentage of hyaluronic acid monomer residues having the azide functionality.

Each R⁵ substituent is hydrogen or a ligand comprising a peptide. In some examples, each R⁵ is hydrogen. In other examples, at least at least some of the R⁵ substituents are the ligand comprising the peptide. The peptide can be a cell-instructive peptide or a pro-regenerative peptide having from 3-100 (e.g., 3-80, 3-50, 30-20, 10-20, 10-12) amino acids. The ligand comprising the peptide can have the structure:

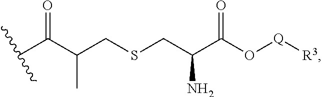

where Q is an optional spacer, and R³ is the peptide. Q in some instances is present in the ligand and has the structure:

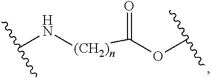

where n is an integer ranging from 1 to 10, e.g., 2-8, 3-6, or 5. The azide-modified hyaluronic acid polymer can be covalently attached to the peptide first by methacrylating free-OH groups on the polymer, which can react with a terminal cysteine residue on the ligand (optionally separated from the peptide by a spacer, Q) through thiol-Michael Addition.

The azide-modified hyaluronic acid polymer can be present in the aqueous solution at a concentration of 1-10% weight by volume. For instance, methacrylated hyaluronic acid polymer can be present in the aqueous solution at a concentration of 1-10%, 1-8%, 1-5%, or about 4% (where “about” means plus and minus 10% of the stated value) weight by volume.

The crosslinker can be present in similar concentrations. In some examples, the crosslinker is water soluble and thus applied to the joint tissue in an aqueous solution. Other polar aprotic solvents can be used to deliver the crosslinker such as DMF or DMSO. The crosslinker has at least two alkyne functional groups to facilitate crosslinking two or more separate hyaluronic polymer chains within the joint tissue. Examples include various crosslinkers having polyethylene glycol (PEG) residues to impart polarity or water-solubility. Specific examples include crosslinkers having the structure:

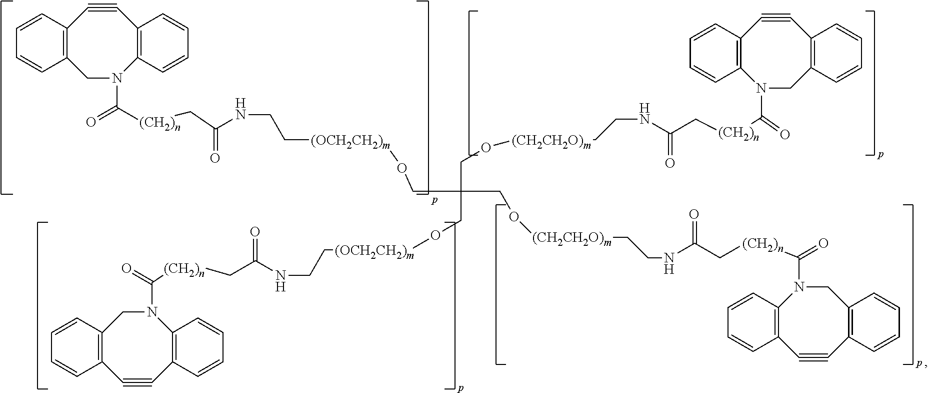

where n and m are independently integers ranging from 1-12 (e.g., 2-10, 3-8, 3-6,2-4), and p is an integer that is 1 (a four-arm PEG-dibenzocyclooctyne,-DBCO crosslinker) or 2 (an eight-arm PEG-DBCO crosslinker). Other examples include 2-arm versions, e.g., a crosslinker having DBCO-PEG linker-DBCO structure, similar to the 4- and 8-arm versions shown in the structure above. These types of PEG-DBCO crosslinkers can have any suitable molecular weight, including a weight-average molecular weight (Mw) of 1-5 kDa as determined by SEC.

### Cell-Instructive and Pro-Regenerative Peptides

As discussed above, each R² substituent on the methacrylated hyaluronic acid polymer, and each R⁵ substituent on the azide-modified hyaluronic acid polymer, can be a ligand comprising a peptide. The peptide can be a cell-instructive peptide or a pro-regenerative peptide having from 3-100 (e.g., 3-80, 3-50, 30-20, 10-20, 10-12) amino acids. Various methods can be used to covalently attach the cell-instructive peptide or the pro-regenerative peptide to the hyaluronic acid polymers. In some examples, the cell-instructive peptide or the pro-regenerative peptide has a terminal cysteine residue, which can react with methacrylate groups on the methacrylated hyaluronic acid polymer through thiol-Michael Addition.

Alternatively, any cell-instructive or pro-regenerative peptide can be modified with a terminal cysteine residue and attached to the methacrylated hyaluronic acid polymer in the same way. An example of a cysteine-based linker is shown below:

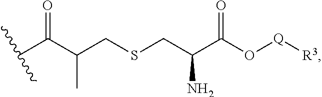

where Q is an optional spacer, and R³ is the cell-instructive or pro-regenerative peptide. Q in some instances is present in the ligand and has the structure:

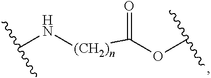

where n is an integer ranging from 1 to 10, e.g., 2-8, 3-6, or 5.

Peptide conjugation strategies can be similarly used with the azide-modified hyaluronic acid polymer. For example, to enable peptide conjugation to the azide-modified hyaluronic acid polymer, free hydroxyl groups on the hyaluronic acid polymer can be methacrylated. Cell-instructive or pro-regenerative peptides can be attached through thiol-Michael Addition as with the methacrylated polymer, e.g., directly through a terminal cysteine residue or by a cysteine linker with the optional spacer between the terminal cysteine residue and the cell-instructive or pro-regenerative peptide.

The terms “cell-instructive peptide” and “pro-regenerative peptide” encompass similar and in some cases overlapping peptide sequences. Both types of peptides aim to influence cellular activities beneficially. The term “cell-instructive peptide” refers to a peptide having 3-100 (e.g., 3-80, 3-50, 30-20, 10-20, 10-12) amino acids which modulates cell behavior through direct signaling interactions. Cell-instructive peptides influence specific cellular behaviors such as adhesion, proliferation, migration, differentiation, or morphogenesis. These peptides can “instruct” cells through biochemical cues-often by mimicking parts of ECM proteins, growth factors, or cell surface ligands. Non-limiting examples of cell-instructive peptides that can be conjugated to the hyaluronic acid polymers include those in Table 1.

TABLE 1
Cell-Instructive Peptides

| Peptide | Function |
|---|---|
| RGD (Arg-Gly-Asp) | Promotes adhesion via integrins (e.g., αvβ3, |
| | α5β1) |
| GHK (Gly-His-Lys) | Wound-healing, repair, anti-inflammatory, |
| | stem cell activation, collagen and |
| | clycosaminoglycan synthesis |
| KLER (Lys-Leu-Glu-Arg)(SEQ ID NO: 1) | Collagen binding and ECM organization, |
| | cell adhesion and migration, tissue-specific |
| | signaling |
| DGEA (Asp-Gly-Glu-Ala)(SEQ ID NO: 2) | Binds to α2β1 integrin; osteogenic |
| | applications |
| PHSRN (Pro-His-Ser-Arg-Asn)(SEQ ID NO: 3) | Synergistic with RGD for integrin binding |
| YIGSR (Tyr-Ile-Gly-Ser-Arg)(SEQ ID NO: 4) | Promotes cell adhesion, inhibits metastasis |
| IKVAV (Ile-Lys-Val-Ala-Val)(SEQ ID NO: 5) | Neurite outgrowth, neural differentiation |
| GFOGER (Gly-Phe-Hyp-Gly-Glu-Arg)(SEQ | Integrin binding, cell adhesion and |
| ID NO: 6) | spreading, osteogenic and chondrogenic |
| | differentiation, tissue regeneration |
| LAVFGRV (Leu-Ala-Val-Phe-Gly-Arg-Val) | Interacts with TGF-β receptors and modulate |
| (SEQ ID NO: 7) | downstream signaling pathways |
| LRKKLGKA (Leu-Arg-Lys-Lys-Leu-Gly-Lys- | Membrane binding and permeabilization, |
| Ala)(SEQ ID NO: 8) | cell adhesion and uptake enhancement |
| HAVDIGGGC (His-Ala-Val-Asp-Ile-Gly-Gly- | Mimics N-Cadherin adhesion motif, stem |
| Gly-Cys)(SEQ ID NO: 9) | cell differentiation and survival, cell |
| | signaling modulation |
| FQSVQDYQAL (Phe-Gln-Ser-Val-Gln-Asp- | Stimulates fibroblast activity and |
| Tyr-Gln-Ala-Leu)(SEQ ID NO: 10) | extracellular matrix formation, mimicking |
| | TGF-β-induced effects in tissue regeneration |
| SGRKSSKMQA (Ser-Gly-Arg-Lys-Ser-Ser- | Mimics histone function, cell instruction, |
| Lys-Met-Gln-Ala)(SEQ ID NO: 11) | nuclear localization |
| GCGYGRGDSPG (Gly-Cys-Gly-Tyr-Gly- | Cell adhesion, migration, and survival by |
| Arg-Gly-Asp-Ser-Pro-Gly)(SEQ ID NO: 12) | mimicking fibronectin's RGD motif |
| ACESPLKRQCGGGS (Ala-Cys-Glu-Ser-Pro- | Cell adhesion, migration, and differentiation |
| Leu-Lys-Arg-Gln-Cys-Gly-Gly-Gly-Ser)(SEQ | by engaging non-integrin receptors. |
| ID NO: 13) | |
| KYPQTLRHVWSYGLR (Lys-Tyr-Pro-Gln- | Mimics TGF-β1 activity, influencing cell |
| Thr-Leu-Arg-His-Val-Trp-Ser-Tyr-Gly-Leu- | migration, differentiation, and collagen |
| Arg)(SEQ ID NO: 14) | synthesis |

The term “pro-regenerative peptide” refers to a peptide having 3-100 (e.g., 3-80, 3-50, 30-20, 10-20, 10-12) amino acids which enhances tissue protection, healing, and repair processes. Pro-regenerative peptides can mimic the body's natural processes and signaling mechanisms that lead to tissue repair and regeneration. Characteristics and functions of pro-regenerative peptides include cell-signaling, angiogenesis, collagen synthesis, inflammation modulation, stem cell activation, and antioxidant effects. Examples of pro-regenerative peptides include ECM-mimetic peptides, growth-factor peptides, matrix metalloproteinase inhibitor peptides, and Thymosin Beta-4.

Non-limiting examples of pro-regenerative peptides that can be conjugated to the hyaluronic acid polymers include those in Table 2. Some of the peptide sequences below overlap with or are the same as the exemplary cell-instructive peptides, and thus the terms “cell-instructive peptide” and “pro-regenerative peptide” can in some instances include peptide sequences that can be classified as both types of peptides. In addition, the hyaluronic acid polymers can include any combination of different cell-instructive or pro-regenerative peptides and any combination both cell-instructive and pro-regenerative peptides.

TABLE 2
Pro-Regenerative Peptides

| Peptide | Function |
|---|---|
| RGD (Arg-Gly-Asp) | Promotes adhesion via integrins (e.g., αvβ3, |
| | α5β1) |
| RGDS (Leu-Arg-Gly-Asp-Ser)(SEQ ID | Enhances cell attachment, healing response, |
| NO: 15) | supports vascularization |
| KGHK (Lys-Gly-His-Lys)(SEQ ID NO: 16) | Stimulates collagen production |
| YIGSR (Tyr-Ile-Gly-Ser-Arg)(SEQ ID NO: 4) | Promotes cell adhesion, inhibits metastasis |
| IKVAV (Ile-Lys-Val-Ala-Val)(SEQ ID NO: 5) | Neurite outgrowth, neural differentiation |
| PHSRN (Pro-His-Ser-Arg-Asn)(SEQ ID NO: 3) | Synergistic with RGD for integrin binding |
| CSGGYS (Cys-Ser-Gly-Gly-Tyr-Ser)(SEQ ID | Cell adhesion, ECM interaction, signal |
| NO: 17) | modulation, structural stabilization |
| SVVYGLR (Ser-Val-Val-Tyr-Gly-Leu-Arg) | Promotes mesenchymal cell migration, |
| (SEQ ID NO: 18) | angiogenesis, and tissue remodeling |
| EQRPGRGFSPF (Glu-Gln-Arg-Pro-Arg-Gly- | Cell adhesion and migration, growth factor |
| Phe-Ser-Pro-Phe)(SEQ ID NO: 19) | mimicry |

### Compositions, Systems, and Kits for Protecting, Improving, and Repairing Joint Tissue

The above-described hyaluronic acid polymers can be part of a composition, system, or kit useful for joint tissue preservation, protection, and repair. A composition comprising the methacrylated hyaluronic acid polymer includes the polymer having the structure below:

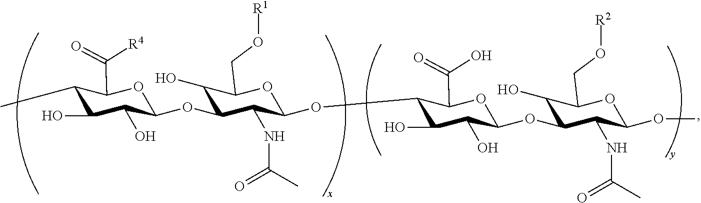

where x and y are independently integers ranging from 5 to 50. The units x and y correspond to molecular weights of the methacrylated hyaluronic acid polymer of 3,000 kDa to 50,000 kDa, e.g., 10,000 kDa to 40,000 kDa, or about 20,000 kDa (where the term “about” indicates the stated value plus and minus 20% of the stated value). The term “molecular weight” refers to weight-average molecular weight (Mw), determined by size exclusion chromatography (SEC).

Each R¹ substituent is hydrogen or a methacrylate group having the structure:

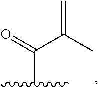 [SMILES: *C(=O)C(=C)C]

At least some of the R¹ substituents are the methacrylate group, which allows for the hyaluronic acid polymer to be photocrosslinked. The methacrylated hyaluronic acid polymer can have a 20% to 100% degree of methacrylation. The “degree of methacrylation” means the percentage of hyaluronic acid monomer residues in the polymer that include methacrylated groups.

Each R² substituent is hydrogen or a ligand comprising a peptide. In some examples, each R² is hydrogen. In other examples, at least at least some of the R² substituents are the ligand comprising the peptide. The peptide can be a cell-instructive peptide or a pro-regenerative peptide having from 3-100 (e.g., 3-80, 3-50, 30-20, 10-20, 10-12) amino acids, including any specific peptide or combination of peptides as described above. The ligand comprising the peptide can have the structure:

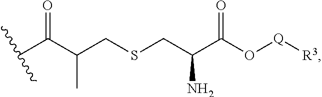

where Q is an optional spacer, and R³ is the peptide. Q in some instances is present in the ligand and has the structure:

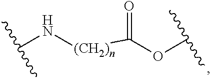

where n is an integer ranging from 1 to 10, e.g., 2-8, 3-6, or 5. The methacrylated hyaluronic acid polymer can be covalently attached to the peptide by the addition of a terminal cysteine residue on the ligand (optionally separated from the peptide by a spacer, Q), which can react with methacrylate groups on the polymer through thiol-Michael Addition.

A system or kit comprising the methacrylated hyaluronic acid polymer can include the polymer in an aqueous solution, e.g., with the methacrylated hyaluronic acid polymer present in the aqueous solution at a concentration of 1-10% weight by volume. The system or kit comprising the methacrylated hyaluronic acid polymer can also include a photoinitiator as discussed above, which can be separate from the hyaluronic acid polymer or in an aqueous solution of the hyaluronic acid polymer. The system or kit can also include instructions for using the hyaluronic acid polymer (e.g., instructions for crosslinking the polymer), as well as other ancillary equipment such as a light source for photo-crosslinking the polymer.

The composition for chemical crosslinking via for example intra-articular injection of the hyaluronic acid polymer and crosslinker can include the azide-modified hyaluronic acid polymer having the structure:

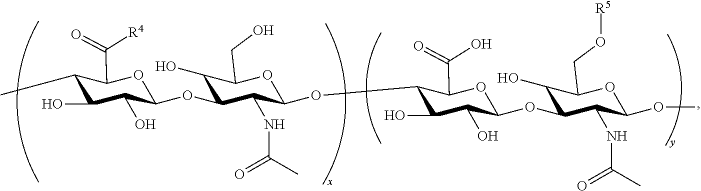

where x and y are independently integers ranging from 5 to 50, generally corresponding to a weight-average molecular weight (Mw) as determined by SEC of 3,000 kDa to 50,000 kDa, e.g., 10,000 kDa to 40,000 kDa, or about 20,000 kDa (where the term “about” indicates the stated value plus and minus 20% of the stated value). The azide-modified hyaluronic acid polymer can have a 20% to 100% degree of azide modification, defined as the percentage of hyaluronic acid monomer residues having the azide functionality.

Each R⁴ substituent is-OH or an azide group having the structure:

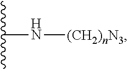

where n is an integer ranging from 1-10, e.g., 2-8, 3-6, or 4. At least some of the R⁴ substituents are the azide group, which undergoes chemical crosslinking with the alkyne functionalized crosslinker through Click chemistry.

Each R⁵ substituent is hydrogen or a ligand comprising a peptide. In some examples, each R⁵ is hydrogen. In other examples, at least at least some of the R⁵ substituents are the ligand comprising the peptide. The peptide can be a cell-instructive peptide or a pro-regenerative peptide having from 3-100 (e.g., 3-80, 3-50, 30-20, 10-20, 10-12) amino acids, including any specific peptide(s) described above. The ligand comprising the peptide can have the structure:

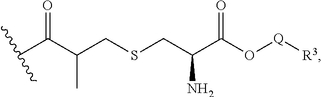

where Q is an optional spacer, and R³ is the peptide. Q in some instances is present in the ligand and has the structure:

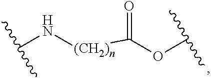

where n is an integer ranging from 1 to 10, e.g., 2-8, 3-6, or 5. The azide-modified hyaluronic acid polymer can be covalently attached to the peptide first by methacrylating free-OH groups on the polymer, which can react with a terminal cysteine residue on the ligand (optionally separated from the peptide by a spacer, Q) through thiol-Michael Addition.

A system or kit comprising the azide-modified hyaluronic acid polymer can include the polymer in an aqueous solution, e.g., with the azide-modified hyaluronic acid polymer present in the aqueous solution at a concentration of 1-10% weight by volume. The azide-modified hyaluronic acid polymer can be present in the aqueous solution at a concentration of 1-10% weight by volume. For instance, methacrylated hyaluronic acid polymer can be present in the aqueous solution at a concentration of 1-10%, 1-8%, 1-5%, or about 4% (where “about” means plus and minus 10% of the stated value) weight by volume.

The system or kit can also include the crosslinker having at least two alkyne groups which can react with the azide groups of the hyaluronic acid polymer. The crosslinker can be provided neat or as a solid or can be in a solution (e.g., an injectable solution separate from an injectable solution of the azide-modified hyaluronic acid polymer). In some examples, the crosslinker is water soluble and thus can be present in an aqueous solution. Other polar aprotic solvents can be used to dissolve the crosslinker include DMF or DMSO. Examples include various crosslinkers having polyethylene glycol (PEG) residues to impart polarity or water-solubility. Specific examples include crosslinkers having the structure:

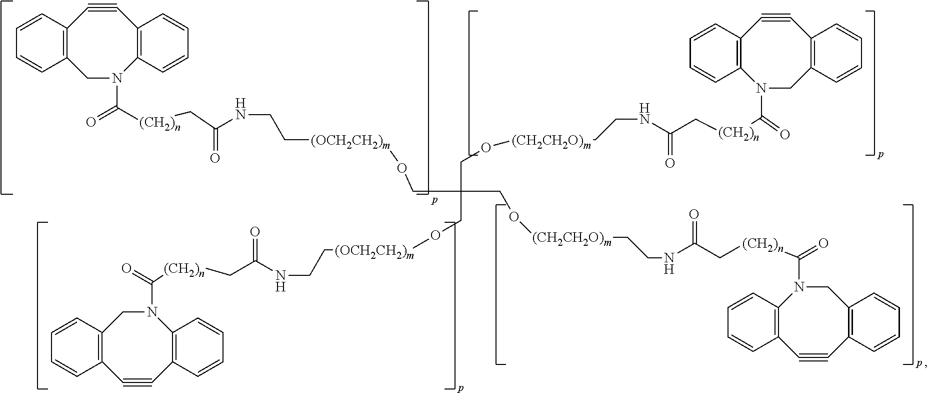

where n and m are independently integers ranging from 1-12 (e.g., 2-10, 3-8, 3-6,2-4), and p is an integer that is 1 (a four-arm PEG-dibenzocyclooctyne,-DBCO crosslinker) or 2 (an eight-arm PEG-DBCO crosslinker). The system or kit can also include instructions for using the azide-modified hyaluronic acid polymer (e.g., instructions for crosslinking the polymer), as well as other ancillary equipment including surgical equipment.

### EXAMPLES

The following examples further illustrate this disclosure. The scope of the disclosure and claims is not limited by the scope of the following examples.

### Example 1 Modified HA Integrates into Cartilage Tissue at Lower Molecular Weights

Methods: Hyaluronic acid (HA; 20, 75, 100 kDa) was methacrylated with methacrylic anhydride (50% modification). Methacrylated HA (20, 75, 100 kDa) was applied to digested cartilage explants (0.1% collagenase for 30 minutes; to mimic OA), with the hydrogel precursor solution also containing Lithium Phenyl (2,4,6-Trimethylbenzoyl) Phosphinate (LAP; for crosslinking) and methacrylated rhodamine (for visualization). Hydrogel precursor solution was allowed 5 minutes to diffuse into cartilage, followed by 5 minutes of blue light crosslinking (400-500 nm, 20 mW/cm²). Explants were rinsed and subject to creep indentation testing. After mechanical testing, explants were embedded in OCT (optimal cutting temperature), cryo-sectioned, and cross-sections were visualized for depth and intensity of material integration. The 20 kDa methacrylated HA integrated the best into cartilage tissue, both in terms of intensity and depth. The 20 kDa and 75 kDa also provided the best reinforcement of cartilage mechanical properties.

### Example 2 Modified HA Integrates into Joint Tissues

Methods: Methacrylated HA (20 kDa), with LAP and methacrylated rhodamine, was applied to a rat knee joint, allowed 5 minutes for diffusion, and photocrosslinked with blue light. Joint tissues were harvested, rinsed, and imaged via confocal microscopy to assess integration. Integration into both the tibial plateau and femoral condyle cartilage surfaces was verified. Furthermore, modified HA integrated into the meniscus and synovium and fat pad as well. These results demonstrate that the HA can penetrate and integrate with multiple tissues across the joint.

### Example 3—Ilantide Peptide Conjugation Can Reduce Inflammatory Activity

Methods: Methacrylated hyaluronic acid (HA; 75 kDa) was conjugated with a cysteine-augmented Ilantide peptide (anti-inflammatory; C-{6-Aminohexanoic acid “Ahx”}-SGRKSSKMQA (SEQ ID NO:11)) via a Michael addition. To investigate how the Ilantide peptide conjugation could aid in reducing inflammation, we embedded bovine chondrocytes into either MeHA hydrogels or MeHA-Ilantide hydrogels for 24 hours, followed by application of IL-1β for 24 hours. Constructs were stained for NF-kB for quantification of fluorescence per cell. Chondrocytes in gels with Ilantide peptide showed less NF-kB cellular staining in the presence of IL-1β, returning to near control levels. These results demonstrate successful conjugation and activity of the peptide in lowering inflammatory response.

### Example 4—Initial Formulations of Injectable Version

Methods: Hyaluronate-azide (50% modification, 20 kDa) was mixed with either DBCO-PEG-DBCO (2 kDa) or 4-arm PEG-DBCO (5 kDa) in 1:1, 1:4, 4:1 ratios of azides to DBCO groups. Hydrogels were formed and tested with nanoindentation, applying a 0.IN peak load to obtain a Hertzian modulus. Hydrogels with the 4-arm PEG-DBCO outperformed the “2-arm” version in terms of mechanical stiffness. For the 4-arm PEG-DBCO, a 1:1 ratio resulted in a modulus of ˜2 kPa, and a 1:4 ratio resulted in a modulus of ˜4 kPa. These results indicate that hydrogel ratios can be tuned for optimized gelation of the components.

### Example 5—Integration of Injectable Version into Cartilage

Methods: HA-Azide (4% w/v) was premixed with DBCO-488 for fluorescent visualization. This solution was applied to the surface of a bovine cartilage explant and allowed 5 minutes to diffuse into the tissue. Next, a solution of DBCO-PEG-DBCO (4% w/v) was applied to the surface and given 10 minutes to diffuse and react with the HA. Explants were rinsed in PBS to remove noncrosslinked gel components, and explants were embedded in OCT for cross-sectional imaging with confocal microscopy. The HA-azide integrated with first few hundred microns of cartilage, like the prior light-based system. The results demonstrate the ability of the injectable version to integrate with cartilage tissue.

### Example 6—Integration of Injectable Version into Rat Knee Joints

Methods: HA-Azide (4% w/v), premixed with DBCO-555 was injected into a rat knee joint, given 5 minutes to diffuse into tissues. Then 4-arm PEG-DBCO was injected into joints and allowed 10 minutes to crosslink. Rat joints were disarticulated, rinsed in PBS to remove noncrosslinked components and visualized by eye for red staining. Upon opening the joint and rinsing, the medial femoral condyle and tibial plateau surfaces exhibited a red tint, indicating material integration. Additionally, red stain was shown in the other tissues of the joint, including the synovium and meniscus. These results provide feasibility of the injectable approach to integrate into joint tissues.

### Example 7—Other Peptides and Formulation of Hydrogels (Prophetic in Part)

Methods: Thiolated peptides were synthesized for conjugation to HA. These include TGF-β mimetic (C {Ahx} ACESPLKRQCGGGS (SEQ ID NO:13)), HAVDI (HAVDIGGGC (SEQ ID NO:9)), RGD (GCGYGRGDSPG (SEQ ID NO:12)), as cell-instructive peptides. These peptides were successfully synthesized. For eventual combination of an injectable and peptide-delivering gels, sodium hyaluronate will be functionalized with both azide and methacrylate groups. Peptides will be conjugated to methacrylate groups via Michael addition. This precursor solution will then be injected first, followed by injection of a DBCO-PEG molecule (2-arm, 4-arm, or 8-arm) for crosslinking to occur.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

What is claimed is:

1. A method of protecting or improving the quality of joint tissue in a subject, comprising:
a) applying an aqueous solution of a methacrylated hyaluronic acid polymer to the joint tissue of the subject, wherein the methacrylated hyaluronic acid polymer has the structure: 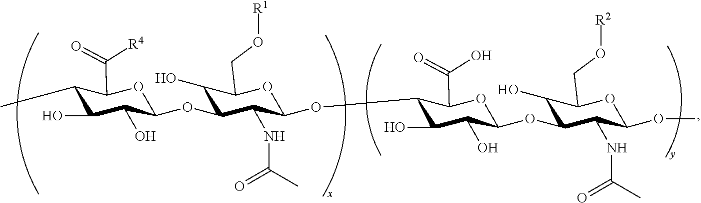
wherein x and y are independently integers ranging from 5 to 50;
each R¹ substituent is hydrogen or a methacrylate group having the structure: 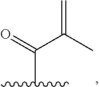 [SMILES: *C(=O)C(=C)C]
wherein at least some of the R¹ substituents are the methacrylate group; and
each R² substituent is hydrogen or a ligand comprising a peptide;
b) allowing the aqueous solution to diffuse into the joint tissue; and
c) photo-crosslinking the methacrylated hyaluronic acid polymer to form a hydrogel within the joint tissue.

2. The method of claim 1, wherein at least some of the R² substituents are the ligand comprising the peptide.

3. The method of claim 1, wherein the ligand comprising the peptide has the structure: 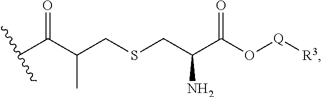
wherein Q is an optional spacer, and
R³ is the peptide.

4. The method of claim 3, wherein Q has the structure: 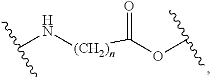
wherein n is an integer ranging from 1 to 10.

5. The method of claim 1, wherein the peptide is a cell-instructive peptide or a pro-regenerative peptide having from 3-100 amino acids.

6. The method of claim 1, wherein the methacrylated hyaluronic acid polymer has a molecular weight of 3,000 kDa to 50,000 kDa.

7. The method of claim 1, wherein the methacrylated hyaluronic acid polymer has a 20% to 100% degree of methacrylation.

8. The method of claim 1, wherein the methacrylated hyaluronic acid polymer is present in the aqueous solution at a concentration of 1-10% weight by volume.

9. The method of claim 1, wherein the aqueous solution further comprises a photoinitiator.

10. A method of repairing damaged articular cartilage in joint tissue of a subject, comprising:
a) microfracturing a portion of the subchondral bone plate beneath a defect area of the damaged articular cartilage and allowing a fibrin clot to form in the defect area;
b) applying an aqueous solution of an azide-modified hyaluronic acid polymer to the joint tissue of the subject, wherein the azide-modified hyaluronic acid polymer has the structure: 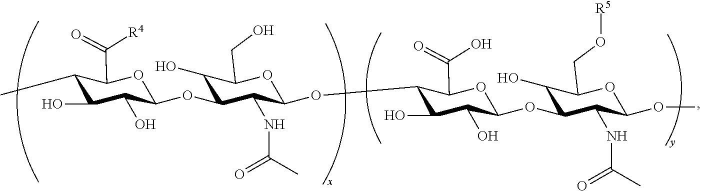
wherein x and y are independently integers ranging from 5 to 50;
each R⁴ substituent is-OH or an azide group having the structure: 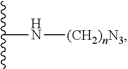
wherein n is an integer ranging from 1-10,
wherein at least some of the R⁴ substituents are the azide group, and
each R⁵ substituent is hydrogen or a ligand comprising a peptide;
c) allowing the aqueous solution to diffuse into the defect area of the damaged articular cartilage;
d) applying a solution of a crosslinker to the joint tissue of the subject, wherein the crosslinker has at least two alkyne functional groups; and
e) allowing the azide-modified hyaluronic acid polymer to react with the alkyne functional groups of the crosslinker via cycloaddition within the joint tissue to form a hydrogel.

11. The method of claim 10, wherein the hydrogel infiltrates the fibrin-rich clot.

12. The method of claim 10, wherein the aqueous solution of the azide-modified hyaluronic acid polymer and the solution of the crosslinker are applied to the joint tissue of the subject by intra-articular injection.

13. The method of claim 10, wherein at least some of the R⁵ substituents are the ligand comprising the peptide.

14. The method of claim 10, wherein the ligand comprising the peptide has the structure: 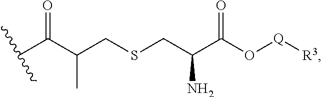
wherein Q is an optional spacer, and
R³ is the peptide.

15. The method of claim 14, wherein Q has the structure: 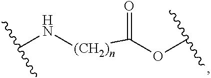
wherein n is an integer ranging from 1 to 10.

16. The method of claim 10, wherein the peptide is a cell-instructive peptide or a pro-regenerative peptide having from 3-100 amino acids.

17. The method of claim 10, wherein the azide-modified hyaluronic acid polymer has a molecular weight of 3,000 kDa to 50,000 kDa.

18. The method of claim 10, wherein the azide-modified hyaluronic acid polymer has a 20% to 100% degree of azide modification.

19. The method of claim 10, wherein the azide-modified hyaluronic acid polymer is present in the aqueous solution at a concentration of 1-10% weight by volume.

20. The method of claim 10, wherein the crosslinker has the structure: 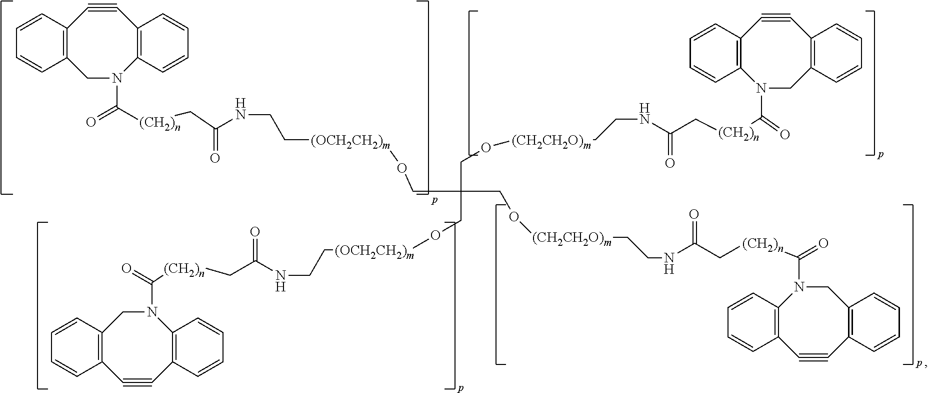
wherein n and m are independently integers ranging from 1-12; and
p is an integer ranging from 1-2.


SEQUENCE LISTING (19 sequences)

SEQ ID NO: 1 - protein, 4 aa, synthetic construct
KLER

SEQ ID NO: 2 - protein, 4 aa, synthetic construct
DGEA

SEQ ID NO: 3 - protein, 5 aa, synthetic construct
PHSRN

SEQ ID NO: 4 - protein, 5 aa, synthetic construct
YIGSR

SEQ ID NO: 5 - protein, 5 aa, synthetic construct
IKVAV

SEQ ID NO: 6 - protein, 6 aa, synthetic construct
GFOGER

SEQ ID NO: 7 - protein, 7 aa, synthetic construct
LAVFGRV

SEQ ID NO: 8 - protein, 8 aa, synthetic construct
LRKKLGKA

SEQ ID NO: 9 - protein, 9 aa, synthetic construct
HAVDIGGGC

SEQ ID NO: 10 - protein, 10 aa, synthetic construct
FQSVQDYQAL

SEQ ID NO: 11 - protein, 10 aa, synthetic construct
SGRKSSKMQA

SEQ ID NO: 12 - protein, 11 aa, synthetic construct
GCGYGRGDSPG

SEQ ID NO: 13 - protein, 14 aa, synthetic construct
ACESPLKRQCGGGS

SEQ ID NO: 14 - protein, 15 aa, synthetic construct
KYPQTLRHVWSYGLR

SEQ ID NO: 15 - protein, 4 aa, synthetic construct
RGDS

SEQ ID NO: 16 - protein, 4 aa, synthetic construct
KGHK

SEQ ID NO: 17 - protein, 6 aa, synthetic construct
CSGGYS

SEQ ID NO: 18 - protein, 7 aa, synthetic construct
SVVYGLR

SEQ ID NO: 19 - protein, 11 aa, synthetic construct
EQRPGRGFSPF